(19) United States
(12) Patent Application Publication
(10) Pub. No.: US 2021/0381080 A1
(43) Pub. Date: Dec. 9, 2021

(54) REAGENT COMPOSITIONS FOR METAL SOLVENT EXTRACTION AND METHODS OF PREPARATION AND USE THEREOF

(71) Applicant: BASF SE, LudwigshafenamRhein (DE)

(72) Inventor: Jack Bender, Tucson, AZ (US)

(21) Appl. No.: 17/282,489

(22) PCT Filed: Oct. 10, 2019
(86) PCT No.: PCT/US19/55502

§ 371 Date: Apr. 2, 2021

Related U.S. Application Data
(60) Provisional application No. 62/744,804, filed on Oct. 12, 2018.

(51) Int. Cl. C22B 3/30; C07C 251/48; C22B 15/00

(52) U.S. Cl. CPC C22B 3/30; C22B 15/0089; C07C 251/48

(57) ABSTRACT

Disclosed are reagent compositions for metal solvent extraction and methods of preparation and use thereof. In certain embodiments, the disclosure relates to recovering metal values from an aqueous process stream using solvent extraction and employing a mixture of oxime reagents that synergistically improves metal recovery, rate of degradation and/or metal selectivity.

### FIELD

Disclosed herein are reagent compositions for metal solvent extraction and methods of preparation and use thereof. In certain embodiments, the disclosure relates to recovering metal values from an aqueous process stream using solvent extraction and employing a mixture of oxime reagents that synergistically improves metal recovery, rate of degradation and/or metal selectivity.

### BACKGROUND

Copper, copper alloys and numerous other valuable metals have been in use for millennia. Because of the importance of such metals, entities continue to research ways to increase the efficiency and productivity of procurement methods. For example, it is critical for mines to maximize efficiency when extracting metals from ore.

Copper-containing ores are typically classified into two categories—oxidic and sulfidic ores. Oxidic ores (e.g., cuprite, malachite, and azurite) are found near the surface as they are oxidation products of the deeper secondary and primary sulfidic ores (e.g., chalcopyrite, bornite, and chalcocite). Due to the chemical nature of copper oxides and secondary sulfides, mines typically treat the ore with hydrometallurgical processes—i.e., heap leaching, solvent extraction, and electrowinning. Approximately 20% of the world's annual copper production is obtained through hydrometallurgical processes.

During hydrometallurgical processes, metal is extracted when the metal-containing material is leached in one of several ways. Leaching is typically accomplished by applying a lixiviant to a collection of ore. The most common lixiviant used in the mining industry is sulfuric acid (“H₂SO₄”) because it provides efficient and cost effective liberation of the metal from the ore. The leaching process can be a heap, dump, percolation or agitation leaching process.

Reagents used in solvent extraction processes generally have certain characteristics relating to phase separation, reagent stability and rate of reaction. However, under some conditions, known reagents may bind copper very tightly such that only a small portion of the copper can be recovered during stripping. To improve stripping, a thermodynamic modifier can be added to the extractant. Alternatively, extractants can be modified or formulated to improve their stripping properties. However, it has been found that the copper content of the stripped organic may be less than expected.

Reagents can also experience degradation resulting from chemical hydrolysis of a ketone or aldehyde in the compound. The concentration of hydrolysis byproducts in the organic phase increases until the rate of formation equals the rate of loss in entrainment. The rate at which hydrolysis occurs is dependent on the acid concentration and the temperature of the system. Some reagents may not work appropriately as a result of hydrolysis. In certain high temperature processes, the reagent compounds can experience a high rate of degradation and the level of degradation byproducts can be as high as 100% of the reagent concentration in the organics circuit. These byproducts significantly increase the density and viscosity of the organic phase, which in turn causes slower phase disengagement and higher entrainments.

Copper/iron selectivity is also very important in solvent extraction/electrowinning systems. Iron transferred to an electrowinning system has a negative effect on the processing of copper in the electrolyte. As the concentration of ferric ions increases, there is a significant drop in current efficiency. In addition to cost resulting from the drop in current efficiency, there is the additional cost of bleeding the system to control the iron concentration. Bleeding electrolyte results in a reduction of cobalt concentration (in addition to other additives) which is added to protect lead anodes, which can be expensive in an electrowinning plant.

There remains a need for reagent compositions and methods of preparing and using the reagent compositions in a solvent extraction process for recovering metal from ore. For example, there is a need for improved reagent compositions containing compounds that interact synergistically to improve metal recovery, rate of degradation and/or metal selectivity.

### BRIEF SUMMARY

According to embodiments, disclosed herein is a reagent composition comprising a mixture of at least two oximes, at least one first oxime having a structure represented by:

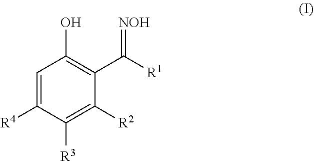

wherein R¹ is hydrogen or a C₁₋₂₂ linear or branched alkyl or alkenyl group,

R², R³ and R⁴ are each independently hydrogen, a halogen, a linear or branched C₁₋₂₂ alkyl group, an —OR⁶ wherein R⁶ is a C₁₋₂₂ linear or branched alkyl group, a C₂₋₂₂ linear or branched alkenyl group, a C₆ aryl group, or a C₇₋₂₂ aralkyl group, wherein at least one of R², R³ or R⁴ is not hydrogen; and

at least one second oxime having a structure represented by:

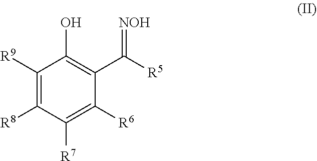

wherein R⁵ is hydrogen or a C₁₋₂₂ linear or branched alkyl or alkenyl group,

R⁶, R⁷ and R⁸ are each independently hydrogen, a halogen, a linear or branched C₁₋₂₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group, wherein at least one of R⁶, R⁷ or R⁸ is not hydrogen, and

R⁹ is a C₁₋₂₂ linear or branched alkyl or alkenyl group, and

wherein when R¹ is hydrogen, the reagent composition comprises less than about 70% by volume of the at least one first oxime,

wherein when R¹ is a C₁₋₂₂ linear or branched alkyl or alkenyl group, the reagent composition comprises less than about 85% by volume of the at least one first oxime,

wherein when R⁵ is hydrogen, the reagent composition comprises at least about 15% by volume to less than about 70% by volume of the at least one second oxime, and

wherein when R⁵ a C₁₋₂₂ linear or branched alkyl or alkenyl group, the reagent composition comprises at least about 15% by volume of the at least one second oxime.

According to further embodiments, disclosed herein is a method comprising

preparing a reagent composition comprising a mixture of at least two oximes, the mixture comprising:

at least one first oxime having a structure represented by:

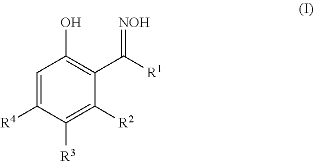

wherein R¹ is hydrogen or a C₁₋₂₂ linear or branched alkyl or alkenyl group,
R², R³ and R⁴ are each independently hydrogen, a halogen, a linear or branched C₁₋₂₂ alkyl group, OR⁶ wherein R⁶ is a C₁₋₂₂ linear or branched alkyl group, a C₂₋₂₂ linear or branched alkenyl group, a C₆ aryl group, or a C₇₋₂₂ aralkyl group, wherein at least one of R², R³ or R⁴ is not hydrogen; and

at least one second oxime having a structure represented by:

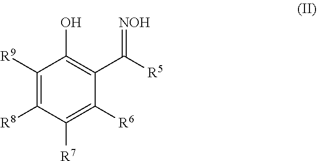

wherein R⁵ is hydrogen or a C₁₋₂₂ linear or branched alkyl or alkenyl group,

R⁶, R⁷ and R⁸ are each independently hydrogen, a halogen, a linear or branched C₁₋₂₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group, wherein at least one of R⁶, R⁷ or R⁸ is not hydrogen, and

R⁹ is a C₁₋₂₂ linear or branched alkyl or alkenyl group.

According to embodiments, described herein is a method of recovering a metal from an aqueous solution comprising metal values, the method comprising:

contacting the aqueous solution with an organic solution comprising an organic solvent and a dissolved reagent composition, wherein the reagent composition comprises a mixture of:

at least one first oxime having a structure represented by:

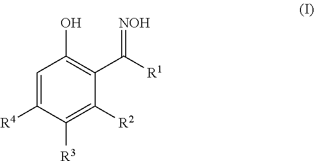

wherein R¹ is hydrogen or a C₁₋₂₂ linear or branched alkyl or alkenyl group,

R², R³ and R⁴ are each independently hydrogen, a halogen, a linear or branched C₁₋₂₂ alkyl group, OR⁶ wherein R⁶ is a C₁₋₂₂ linear or branched alkyl group, a C₂₋₂₂ linear or branched alkenyl group, a C₆ aryl group, or a C₇₋₂₂aralkyl group, wherein at least one of R², R³ or R⁴ is not hydrogen; and

at least one second oxime having a structure represented by:

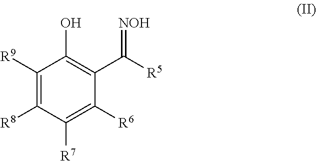

wherein R⁵ is hydrogen or a C₁₋₂₂ linear or branched alkyl or alkenyl group,

R⁶, R⁷ and R⁸ are each independently hydrogen, a halogen, a linear or branched C₁₋₂₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group, wherein at least one of R⁶, R⁷ or R⁸ is not hydrogen, and

R⁹ is a C₁₋₂₂ linear or branched alkyl or alkenyl group.

The above summary provides a basic understanding of the disclosure. This summary is not an extensive overview of all contemplated aspects, and is not intended to identify all key or critical elements or to delineate the scope of any or all aspects of the disclosure. Its sole purpose is to present one or more aspects in a summary form as a prelude to the more detailed description that follows and the features described and particularly pointed out in the claims.

### BRIEF DESCRIPTION OF THE DRAWINGS

The present disclosure is illustrated by way of example, and not by way of limitation, in the figures of the accompanying drawings in which like references indicate similar elements.

FIG. 1 is a flow diagram for a solvent extraction process according to embodiments herein.

FIG. 2A is an Optiform chart for a pregnant leaching solution (PLS) having an intermediate pH of about 1.8 in accordance with embodiments described herein.

FIG. 2B is an chart for a PLS with a high pH of about 2.1 in accordance with embodiments described herein.

FIG. 2C is an chart for a PLS with a low pH of about 1.2 in accordance with embodiments described herein.

FIG. 2D is a graph showing reagent concentration as a function of metal recovery for a comparative reagent at low pH and reagent compositions according to embodiments described herein.

FIG. 2E is a graph showing reagent concentration as a function of metal recovery for a comparative reagent at intermediate pH and reagent compositions according to embodiments described herein.

FIG. 3A shows the reagent degradation for each of four reagent compositions when mixed with a PLS under harsh conditions.

FIG. 3B shows the reagent degradation for each of four reagent compositions when mixed with an electrolyte solution.

FIG. 4 shows the concentration (ppm) of iron in the organic phase as a function of mass percent of copper loading in the organic phase for a comparative reagent and reagent compositions according to embodiments herein.

FIG. 5A is a chart showing the performance of various reagent compositions in a known solvent extraction process.

FIG. 5B is a chart showing the performance of various reagent compositions in a known solvent extraction process.

FIG. 6A is a chart showing the performance of various reagent compositions in a pilot plan process.

FIG. 6B is a chart showing the performance of various reagent compositions in a pilot plan process.

### DETAILED DESCRIPTION

Embodiments are described herein in the context of reagent compositions and methods of making and using such reagent compositions. Those of ordinary skill in the art will recognize that the following description is illustrative only and is not intended to be in any way limiting. Other aspects will readily suggest themselves to those of ordinary skill in the art having the benefit of this disclosure. Reference will now be made in detail to implementations of the example aspects as illustrated in the accompanying drawings. The same reference indicators will be used to the extent possible throughout the drawings and the following description to refer to the same or like items.

### Definitions

Reference throughout the disclosure to terms such as “one embodiment,” “certain embodiments,” “one or more embodiments,” “various embodiments,” “an embodiment” and so forth mean that a particular feature, structure, material, or characteristic described in connection with the embodiment is included in at least one embodiment. Thus, such terms throughout the disclosure are not necessarily referring to the same embodiment. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “a metal” includes a single metal as well as two or more different metals.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything higher than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

The term “NSAO” is used herein interchangeably with 5-nonylsalicylaldoxime, and refers to a compound having the structure:

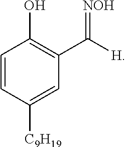

The term “HNAO” is herein used interchangeably with 5-nonyl-2-hydroxyacetophenonoxime, and refers to a compound having the structure:

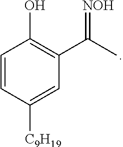

The term “3-MNSAO” is used herein interchangeably with 3-methyl-5-nonylsalicylaldoxime, and refers to a compound having the structure:

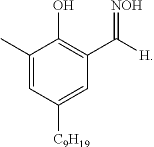

The term “3-MHNAO” is used herein interchangeably with 3-methyl-5-nonyl-2-hydroxyacetophenone oxime, and refers to a compound having the structure:

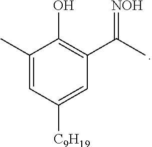

The term “standard oxime” as used herein refers to a compound having a structure represented by:

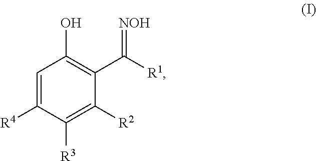

wherein R₁ is hydrogen, a C₁₋₂₂ linear or branched alkyl or alkenyl group, a C₆ aryl group or a C₇₋₂₂ aralkyl group, R²-R⁴ are each independently hydrogen, halogen, a linear or branched C₆₋₁₂ alkyl group, OR⁶ where R⁶ is a C₁₋₂₂ linear or branched alkyl group, a C₂₋₂₂ linear or branched alkenyl group, a C₆ aryl group, or a C₇₋₂₂ aralkyl group and R⁵ is hydrogen, wherein at least one of R²-R⁴ is not hydrogen.

The term “standard ketoxime” as used herein refers to a compound having a structure represented by:

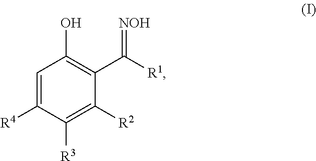

wherein R⁵ is a C₁₋₂₂ linear or branched alkyl or alkenyl group, a C₆ aryl group or a C₇₋₂₂ aralkyl group, R²-R⁴ are each independently hydrogen, halogen, a linear or branched C₆₋₁₂ alkyl group, OR⁶ where R⁶ is a C₁₋₂₂ linear or branched alkyl group, a C₂₋₂₂ linear or branched alkenyl group, a C₆ aryl group, or a C₇₋₂₂ aralkyl group and R⁵ is hydrogen, wherein at least one of R²-R⁴ is not hydrogen.

The term “standard aldoxime” as used herein refers to a compound having a structure represented by:

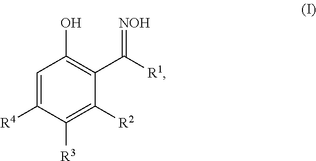

wherein R¹ is hydrogen and R²-R⁴ are each independently hydrogen, halogen, a linear or branched C₆₋₁₂ alkyl group, OR⁶ where R⁶ is a C₁₋₂₂ linear or branched alkyl group, a C₂₋₂₂ linear or branched alkenyl group, a C₆ aryl group, or a C₇₋₂₂ aralkyl group, wherein at least one of R²-R⁴ is not hydrogen.

The term “3-methyl oxime” as used herein refers to a compound having a structure represented by:

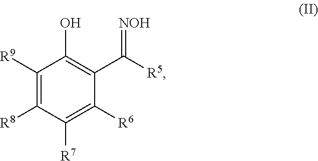

wherein R⁵ is hydrogen, a C₁₋₂₂ linear or branched alkyl or alkenyl group, a C₆ aryl group or a C₇₋₂₂ aralkyl group, R⁶-R⁸ are each independently hydrogen, halogen, a linear or branched C₆₋₁₂ alkyl group, OR¹⁰ where R¹⁰ is a C₁₋₂₂ linear or branched alkyl group, a C₂₋₂₂ linear or branched alkenyl group, a C₆ aryl group, or a C₇₋₂₂ aralkyl group and R⁵ is methyl, wherein at least one of R⁶-R⁸ is not hydrogen, and wherein R⁹ is a C₁₋₂₂ linear or branched alkyl or alkenyl group.

The term “3-methyl ketoxime” as used herein refers to a compound having a structure represented by:

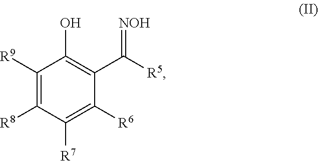

wherein R⁵ is a C₁₋₂₂ linear or branched alkyl or alkenyl group, a C₆ aryl group or a C₇₋₂₂ aralkyl group, R⁶-R⁸ are each independently hydrogen, halogen, a linear or branched C₆₋₁₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group, and R⁹ is methyl, wherein at least one of R⁶-R⁸ is not hydrogen.

The term “3-methyl aldoxime” as used herein refers to a compound having a structure represented by:

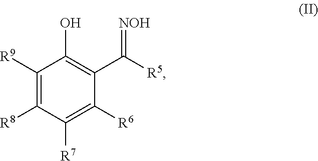

wherein R⁵ is hydrogen, R⁶-R⁸ are each independently hydrogen, halogen, a linear or branched C₆₋₁₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group and R⁹ is methyl, wherein at least one of R⁶-R⁸ is not hydrogen.

The term “mixture of oximes” or “mixture of at least two oximes” as used herein refers to a mixture containing at least one standard oxime and at least one 3-methyl oxime.

The term “reduced consumption” as used herein refers to a decrease in the amount of reagent composition (e.g., a decrease in concentration, a decrease in volume, etc.) needed to achieve a target metal recovery as compared to a comparative reagent.

The term “metal recovery” as used herein refers to the amount by mass (e.g., wt %) of metal recovered from a process stream as compared to the total amount (e.g., wt %) of dissolved metal in the process stream.

The term “degradation” as used herein refers to the acid catalyzed hydrolysis of an oxime to break it down into its constituent components.

The term “metal selectivity” as used herein refers to the likelihood that a reagent will complex with a particular metal over another.

### Reagent Compositions

A reagent composition according to embodiments described herein can include:

a mixture of at least two oximes, at least one first oxime having a structure represented by:

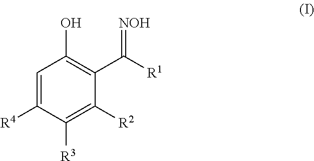

wherein R¹ is hydrogen or a C₁₋₂₂ linear or branched alkyl or alkenyl group,

R², R³ and R⁴ are each independently hydrogen, a halogen, a linear or branched C₁₋₂₂ alkyl group, OR⁶ wherein R⁶ is a C₁₋₂₂ linear or branched alkyl group, a C₂₋₂₂ linear or branched alkenyl group, a C₆ aryl group, or a C₇₋₂₂ aralkyl group, wherein at least one of R², R³ or R⁴ is not hydrogen; and

at least one second oxime having a structure represented by:

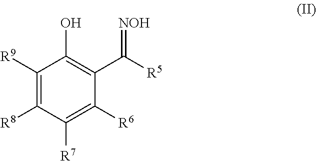

wherein R⁵ is hydrogen or a C₁₋₂₂ linear or branched alkyl or alkenyl group,

R⁶, R⁷ and R⁸ are each independently hydrogen, a halogen, a linear or branched C₁₋₂₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group, wherein at least one of R⁶, R⁷ or R⁸ is not hydrogen, and

R⁹ is a C₁₋₂₂ linear or branched alkyl or alkenyl group.

According to embodiments, when R¹ is hydrogen, the reagent composition can include less than about 70% by volume of the at least one first oxime. In embodiments, when R¹ is a C₁₋₂₂ linear or branched alkyl or alkenyl group, the reagent composition can include less than about 85% by volume of the at least one first oxime. In embodiments, when R⁵ is hydrogen, the reagent composition can include at least about 15% by volume to less than about 70% by volume of the at least one second oxime.

According to various embodiments, the reagent compositions include a mixture of at least two oximes. In some embodiments, the reagent composition includes a standard ketoxime and a 3-methyl ketoxime and/or a 3-methyl aldoxime. In other embodiments, the reagent composition includes a standard aldoxime and a 3-methyl ketoxime and/or a 3-methyl aldoxime. In some embodiments the reagent composition includes a standard ketoxime, a standard aldoxime and a 3-methyl ketoxime and/or a 3-methyl aldoxime.

In one or more embodiments, the reagent composition may contain the standard oxime in an amount of about 25 wt %, about 30 wt %, about 40 wt %, about 50 wt %, about 60 wt %, about 65 wt % about 70 wt %, about 75 wt %, about 80 wt %, or about 85 wt %, or about 1 wt % to about 80 wt %, or about 5 wt % to about 50 wt %, or about 10 wt % to about 40 wt %, or about 20 wt % to about 30 wt % of the total amount of oximes in the reagent composition.

In some embodiments, the reagent composition may contain the 3-methyl oxime in an amount of about 25 wt %, about 30 wt %, about 40 wt %, about 50 wt %, about 60 wt %, about 65 wt % about 70 wt %, about 75 wt %, about 80 wt %, or about 85 wt %, or about 1 wt % to about 80 wt %, or about 5 wt % to about 50 wt %, or about 10 wt % to about 40 wt %, or about 20 wt % to about 30 wt % of the total amount of oximes in the reagent composition.

According to certain embodiments, the reagent composition may contain, in addition to a 3-methyl oxime, both a standard ketoxime and a standard aldoxime at a molar ratio of standard ketoxime to standard aldoxime of about 85:15, or about 60:40, or about 25:75, or about 80:20 to about 30:70, or about 80:20 to about 40:60. According to further embodiments, the reagent composition may contain, in addition to a standard oxime, a 3-methyl ketoxime and a 3-methyl aldoxime at a molar ratio of standard ketoxime to standard aldoxime of about 85:15, or about 60:40, or about 25:75, or about 80:20 to about 30:70, or about 80:20 to about 40:60.

In one or more embodiments, the total concentration of the oximes in the reagent composition can be from about 0.015M to about 3.0M, or about 0.5M to about 2.5M, or about 1.0M to about 2.0M. In certain embodiments, the at least one standard oxime and the at least one 3-methyl oxime can be present in the reagent composition at a weight ratio of the standard oxime to the 3-methyl oxime of about 100:1 to about 1:100, or about 90:1 to about 1:90, or about 80:1 to about 1:80, or about 70:1 to about 1:70, or about 60:1 to about 1:60, or about 50:1 to about 1:50, or about 40:1 to about 1:40, or about 30:1 to about 1:30, or about 20:1 to about 1:20, or about 10:1 to about 1:10, or about 5:1 to about 1:5 or about 2:1 to about 1:2, or about 1:1, or about 1:2, or about 1:5, or about 1:10, or about 1:50, or about 1:100, or about 2:1, or about 5:1, or about 10:1, or about 50:1, or about 100:1.

Reagent compositions according to one or more embodiments include modifiers that can be added to the reagent to increase functionality. U.S. Pat. Nos. 4,978,788; 6,177,055; 6,231,784; 7,585,475 and 7,993,613, the contents of which are incorporated herein by reference, provide examples of modifiers that can be used in accordance with embodiments of the present invention. For example, the use of highly branched chain aliphatic or aliphatic-aromatic C₁₀-C₃₀ esters or C₁₀-C₃₀ alcohols have beneficial results as strip modifiers. Another example is an equilibrium modifier, where the modifier is a linear diester or polyester of an unbranched monocarboxylic acid or unbranched dicarboxylic acid and an unbranched alcohol. One embodiment of the invention is the reagent composition described above, further comprising thermodynamic modifiers. A second embodiment is the reagent composition described above, further comprising kinetic modifiers. Examples of suitable kinetic modifiers include, but are not limited to, dioximes such as 8,9-dioximohexadecane or alpha-bromocarboxylic acids such as alpha-bromolauric acid. In a particular embodiment, the kinetic modifier comprises 5,8-diethyl-7-hydroxydodecan-6-oxime.

The reagent compositions in one or more embodiments include a solvent in which the reagent is dissolved. In one embodiment, the solvent comprises a water immiscible organic solvent. In another embodiment, the water immiscible organic solvent is selected from the group consisting of kerosene, benzene, toluene, xylene and combinations thereof.

The oximes in the composition can be present in any suitable concentration for extraction. For example, in one or more embodiments, concentration of the oxime ranges from about 0.018M to about 1.1M. In specific embodiments, the concentration of oxime ranges from about 0.018M to about 0.9M or 0.018M to about 0.72M. In embodiments in which a ketoxime and aldoxime are present, the concentration of both ketoxime and aldoxime ranges from about 0.018M to about 0.9M or from about 0.018M to about 0.72M. The reagent composition may also be concentrated. Concentrated forms can be useful, for example, for transporting the reagent composition. Several embodiments of concentrated forms can have an oxime concentration of about 1.7M or about 1.8M and up to about 2.25M, 2.5M, and 2.6M. This oxime concentration can be the combined concentration of the oximes in embodiments where more than one oxime is present.

In certain embodiments, the mixture of the reagent composition may include a standard aldoxime, a standard ketoxime and a 3-methyl aldoxime. For example, the mixture may include NSAO, HNAO and 3-MNSAO. The standard aloxime (e.g., NSAO) may be present in an amount of about 0 wt % to about 50 wt %. The standard ketoxime (e.g., HNAO) may be present in an amount of about 20 wt % to about 80 wt %. The 3-methyl aldoxime (e.g., 3-MNSAO) may be present in an amount of about 20 wt % to about 80 wt % of (V). In further examples, the reagent composition may contain a standard aldoxime and a 3-methyl aldoxime. Alternatively, the reagent composition may contain a standard aldoxime, a 3-methyl aldoxime and a 3-methyl ketoxime. For example, the reagent composition may contain about 70 wt % to about 90 wt % of a standard ketoxime (e.g., HNAO), about 3 wt % to about 9 wt % of a 3-methyl aldoxime (e.g., 3-MNSAO) and about 7 wt % to about 21 wt % of a 3-methyl ketoxime (e.g., 3-MHNAO).

According to one or more embodiments, the reagent compositions comprising the mixture of oximes exhibit one or more synergistic effects including reduced consumption, improved metal recovery, improved degradation properties and improved metal selectivity. In one or more embodiments, the formulation of the reagent composition may be based on operating parameters (e.g., pH, temperature, existing extraction agent, etc.) of a known solvent extraction system as will be described in more detail below. In embodiments, the reagent composition may be formulated to contain a mixture of oximes that synergistically achieve a desired result. For example, in certain embodiments, the amount of the reagent composition required to achieve a target recovery of metal from a leaching solution (e.g., sulfuric acid containing metal values) is less than the amount of a comparative reagent (e.g., an existing reagent having a different formulation and that does not comprise the mixture of oximes) needed to achieve the same target recovery. The mixture of oximes in the reagent composition may be chosen to have a synergistic effect on recovery of a metal from a leaching solution such that the mixture of oximes is more effective at lower concentrations to recover the metal than the comparative reagent. The comparative reagent may be, for example, a standard ketoxime, a standard aldoxime, a mixture of two a standard ketoxime and a standard aldoxime, or a mixture of a 3-methyl ketoxime and a 3-methyl aldoxime. In embodiments, the amount of the reagent composition to achieve the target recovery of metal may be at least about 2 wt %, or 3 wt %, or 4 wt %, or 5 wt %, or 6 wt %, or 7 wt %, or 8 wt %, or 9 wt % or 10 wt %, or 11, wt %, or 12 wt %, or 13 wt %, or 14 wt % or 15 wt % less than the amount of the comparative reagent composition needed provide the target recovery.

In further embodiments, at the same concentration of reagent in the leaching solution, the formulation of the reagent composition may be chosen to have a mixture of oximes that provides a higher recovery of metal from the leaching solution than the comparative reagent that does not comprise the mixture of oximes. The mixture of oximes in the reagent composition may be chosen to have a synergistic effect on recovery of a metal from a leaching solution such that the mixture of oximes is more effective at lower concentrations to recover the metal than the comparative reagent. In certain embodiments, the reagent composition provides a recovery of at least about 1 wt %, or 2 wt %, or 3 wt %, or 4 wt %, or 5 wt %, or 6 wt %, or 7 wt %, or 8 wt %, or 9 wt % or 10 wt %, or 11, wt %, or 12 wt %, or 13 wt %, or 14 wt % or 15 wt % higher than the recovery of the comparative reagent.

In further embodiments, the reagent composition may be chosen to have a mixture of oximes such that the composition as a whole has less degradation than a comparative reagent that does not comprise the mixture oximes. The mixture of oximes may interact synergistically to slow degradation of the reagent composition as a whole.

In further embodiments, the reagent composition may be chosen to have a mixture of oximes that, as a whole, have a higher selectivity for a metal (e.g., copper) over another metal (e.g., iron) than a comparative reagent that does not comprise the mixture of oximes. The mixture of oximes may interact synergistically to provides a higher selectivity for a metal over another metal than the comparative reagent.

According to embodiments, the reagent composition includes at least one first oxime having a structure selected from:

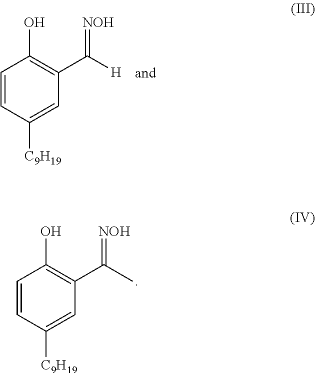

According to embodiments, the reagent composition includes at least one second oxime having a structure selected from:

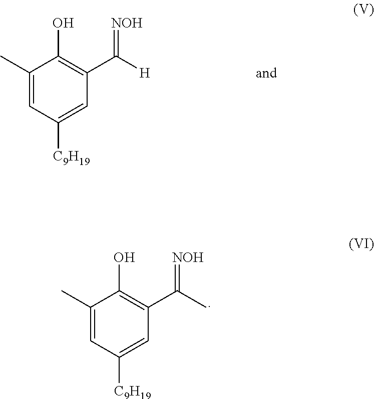

According to embodiments, the reagent composition can include a mixture of oximes as follows:

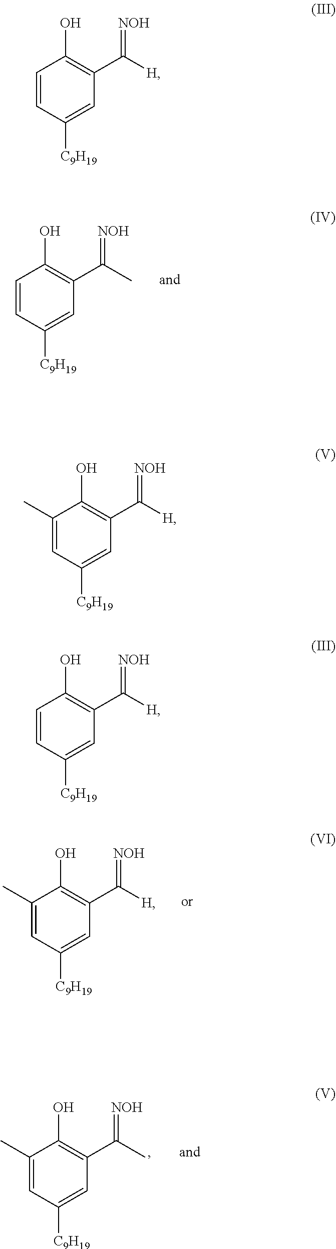

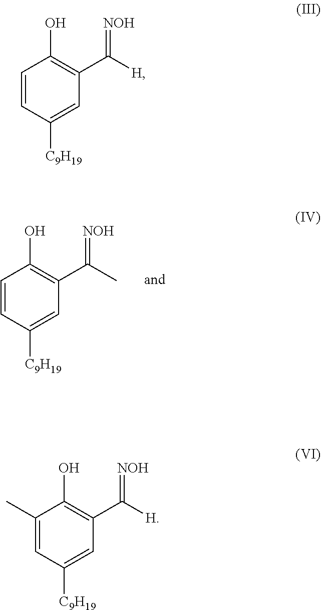

According to embodiments, in the above listed mixtures, mixtures having a compound of structure III (III) can be at a concentration of about 0 wt % to about 50 wt %, mixtures having a compound of structure IV (IV) can be at a concentration of about 20 wt % to about 80 wt %, mixtures having a compound of structure V (V) can be at a concentration of about 20 wt % to about 80 wt %, and mixtures having a compound of structure VI (VI) can be at a concentration of at least about 15 wt %.

According to embodiments, the amount of the reagent composition to provide a target recovery of metal from a process stream is less than an amount of a comparative reagent composition that does not include the mixture. According to embodiments, the amount of the reagent composition to provide the target recovery of metal is at least about 2% by weight less than the amount of the comparative reagent composition to provide the target recovery. In embodiments, the at least about 2% by weight is of a formulated volume of reagent (5.6 max load).

According to further embodiments, when at a target concentration, the reagent composition has a higher recovery of metal from a process stream than a comparative reagent composition that does not comprise the mixture. According to embodiments, the reagent composition provides a recovery of at least about 4% by weight higher than the recovery of the comparative reagent composition.

According to embodiments, the reagent composition can include a mixture of:

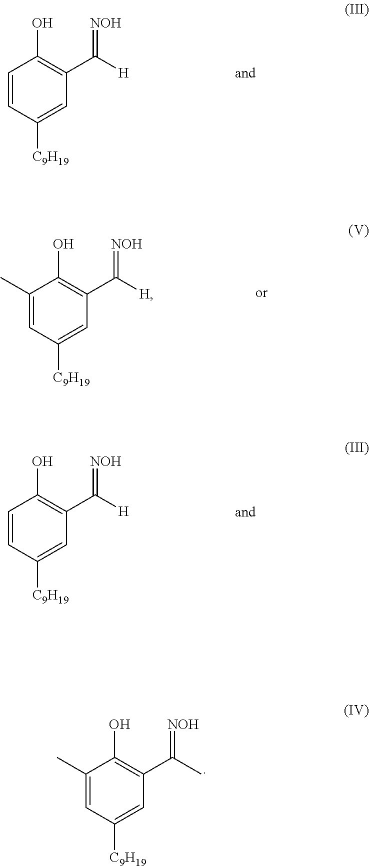

According to embodiments, the reagent composition has less degradation than a comparative reagent composition that does not comprise the mixture.

According to certain embodiments, the reagent composition can include a mixture of oximes as follows:

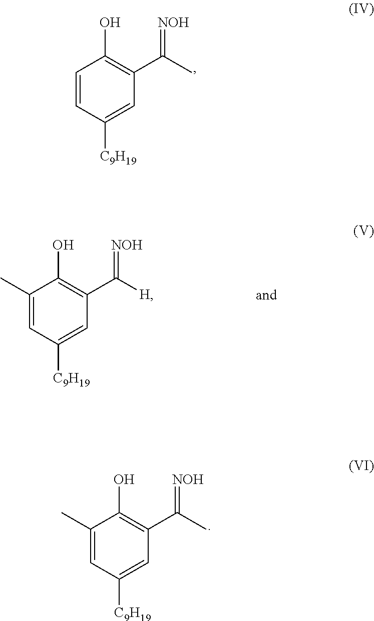

According to embodiments, above-listed mixture can include about 10 wt % to about 70 wt % of compound IV, about 3 wt % to about 85 wt % of compound V and about 7 wt % to about 70 wt % of compound VI.

In certain embodiments, the reagent composition can have a higher selectivity for copper over iron than a comparative reagent composition that does not comprise the mixture. In further embodiments, the comparative reagent composition can include a standard ketoxime, a standard aldoxime, a mixture of two comparative substitutions of the first oxime and a mixture of two comparative substitutions of the second oxime.

### Methods of Preparing the Reagent Compositions

According to various embodiments, described herein are methods of preparing the reagent compositions. The methods include preparing a reagent composition comprising a mixture of at least two oximes as described above.

Methods of making individual oxime compounds are known in the art, such as those disclosed in U.S. Pat. No. 6,632,410, the entire contents of which are incorporated herein by reference. For example, 3-methyl-5-nonylsalicylaldoxime can be made by reacting o-cresol with tripropylene in the presence of an acid catalyst such as AMBERLYST® 15 resin to form 4-nonyl-2-cresol, which is in turn converted to the aldehyde by reaction with para-formaldehyde in the presence of a catalyst such as titanium cresylate. The 3-methyl-5-nonylsalicylaldehyde is then reacted with hydroxylamine sulfate to form the 3-methyl-5-nonylsalicylaldoxime. In all cases, the total number of carbon atoms in all of R²-R⁵ groups must be large enough so that the corresponding copper-extractant complex is soluble in the hydrocarbon solvent.

According to various embodiments, methods for preparing reagent compositions as described herein can include mixing a standard oxime with a 3-methyl oxime. The oximes may be dissolved in an organic solvent, for example, a water immiscible organic solvent. The organic solvent may include, but is not limited to, kerosene, benzene, toluene, xylene and combinations thereof.

The at least one first oxime in the mixture of oximes can be a standard oxime. The at least one second oxime in the mixture of oximes can be a 3-methyl oxime. As discussed above, combining the at least one first oxime with the at least one second oxime to form the reagent composition can result in a synergistic effect between the oxime components. For example, the reagent compositions comprising the mixture of oximes can interact synergistically to provide reduced consumption, improved metal recovery, improved degradation properties and improved metal selectivity as compared to a comparative reagent that does not comprise the mixture of oximes.

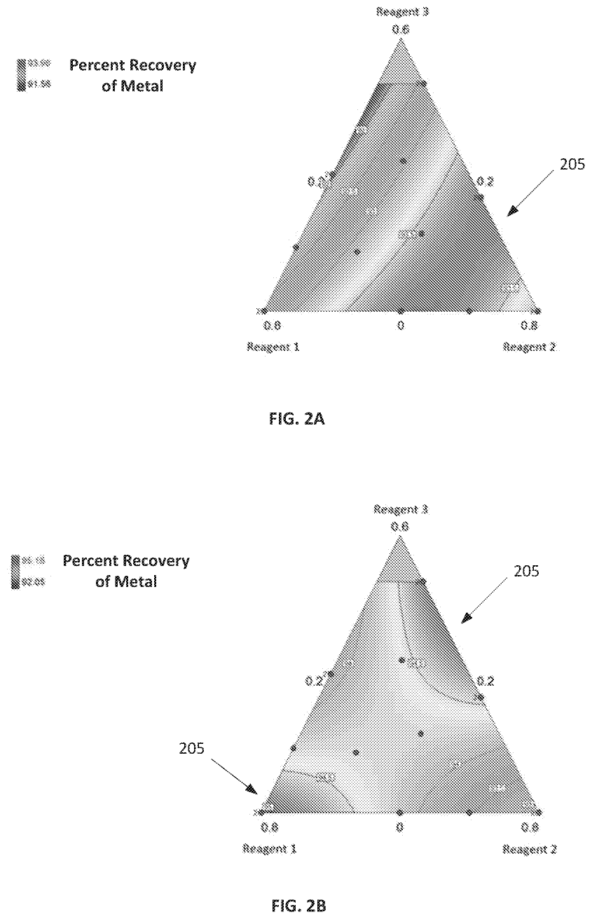

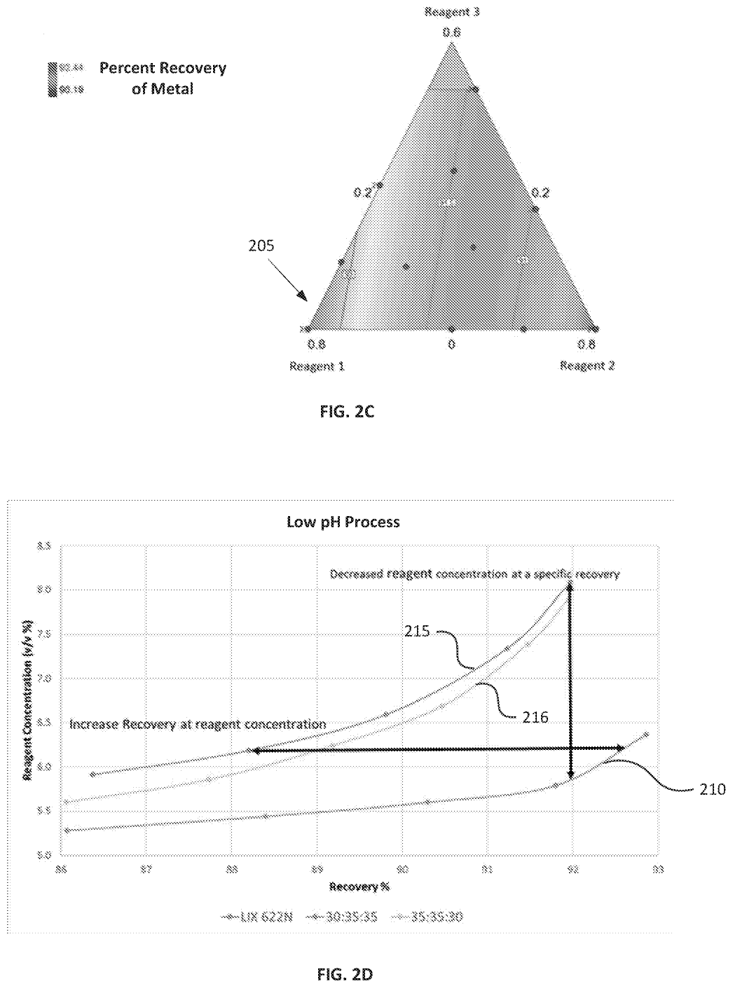

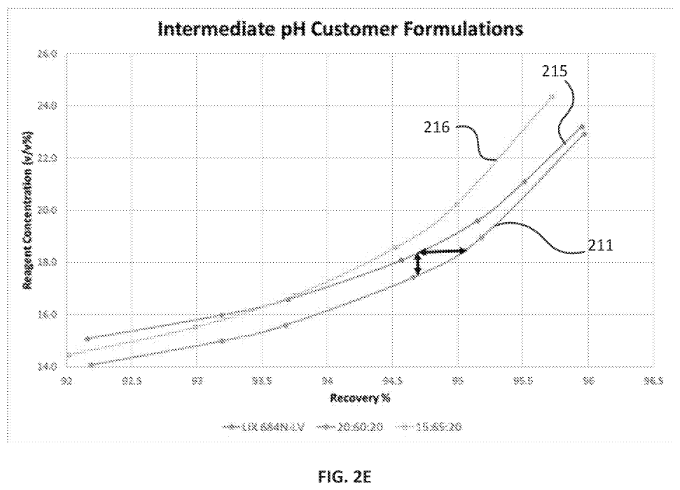

According to one or more embodiments, methods of preparing reagent compositions as described herein can include determining a set of parameters for a formulation blend, for example, using BASF's Optiform process. In determining a set of parameters, a hypothetical parameter or set of parameters are input into one or more computer programs, which then optimizes the parameter or set of parameters to achieve a target recovery of a metal. A statistical program (e.g., StatEase) can be used to determine the composition of the blends and duplicate points for analysis. Once a parameter or set of parameters is chosen, the metal recovery characteristics for a particular formulation comprising a mixture of oximes can be determined by generating appropriate isotherms and using a McCabe Theil modelling program (e.g., BASF's Isocalc®) to calculate the result. In embodiments, mixtures of reagents, for example, in sulfuric acid, can be prepared and the metal recovery for each mixture can be evaluated under various isothermal conditions. The modelling program can include a combination of chemical modeling of extraction and stripping processes and mathematical manipulation of data points. A stripped organic value must be used as a starting point for each extraction isotherm. Once the program has generated either an equilibrium extraction or stripping curve, it allows for the construction of a McCabe-Thiele diagram. The recoveries are then plotted as shown in FIGS. 2A-2C and a hot map is used to determine the formulations with the highest probability of having the highest recovery. As described in more detail in the Examples below, this method can be used to determine a variety of parameters.

Reagents for copper solvent extraction (SX) typically include modified aldoximes, ketoxime reagent, aldoxime/ketoxime blends, and degradation resistant/selective ketoximes and aldoximes. Modified aldoximes, ketoxime and aldoxime/ketoxime blends are ubiquitously used in hydrometallurgy plants around the world. Very selective and degradation resistant aldoximes and ketoximes are also used in hydrometallurgical applications. However, as described in accordance with embodiments herein, these very selective reagents can be blended together with modified aldoximes, ketoxime reagent, aldoxime/ketoxime blends for specialty applications. The resulting reagent compositions according to embodiments can have at least as good copper recoveries while increasing the overall selectivity of copper over iron and providing additional resistance to hydrolytic degradation. The combination of a standard aldoxime and/or standard ketoxime with a 3-methyl aldoxime and/or 3-methyl ketoxime with different properties leads to an offering that can be tailored to the needs of a particular process (e.g., a specific customer's solvent extraction process). The standard aldoxime, standard ketoxime, 3-methyl aldoxime and 3-methyl ketoxime each has different properties as summarized in Table 1.

TABLE 1
Properties of certain oximes

| | Standard | Standard | 3-Methyl | 3-Methyl |
|---|---|---|---|---|
| | Aldoxime | Ketoxime | Aldoxime | Ketoxime |
| Structure | NSAO | HNAO | 3-MNSAO | 3-MHNAO |
| | (Reagent 1) | (Reagent 2) | (Reagent 3) | (Reagent 4) |
| Extraction | Strong | Weak | Very Strong | Moderate |
| Strength | | | | |
| Stripping | Moderate | High | Moderate | High |
| Efficiency | | | | |
| Cu:Fe | High | Low | Very High | Very High |
| Selectivity | | | | |
| Degradation | Poor | High | Very High | Very High |
| Resistance | | | | |
| Kinetics | Fast | Moderate | Fast | Moderate |

In addition to improving metal recovery and removal efficiency (i.e., reducing amount of reagent needed), the reagent compositions can be prepared to improve degradation performance and metal selectivity of copper over iron as compared to a comparative composition. Acid catalyzed hydrolysis (often referred to as degradation) of oximes is accelerated by increases in acid concentration, temperature, polarity of the organic solvent, surface area of the emulsion, coalescence time, structure of the oxime and presence of impurities in both the organic and aqueous phases. As expected, the degradation on the strip side of the SX process is higher than on the extraction side. However, both contribute to the conversion of the oximes to their respective aldehydes and ketones. When degradation rates are high, the subsequent increase in entrainment losses reach an equilibrium which can result in relatively high consumption of extraction reagent. This steady state varies for individual plants, and is the highest for agitation and high temperature leach plants.

Methods of Recovering Metals from Leaching Solutions

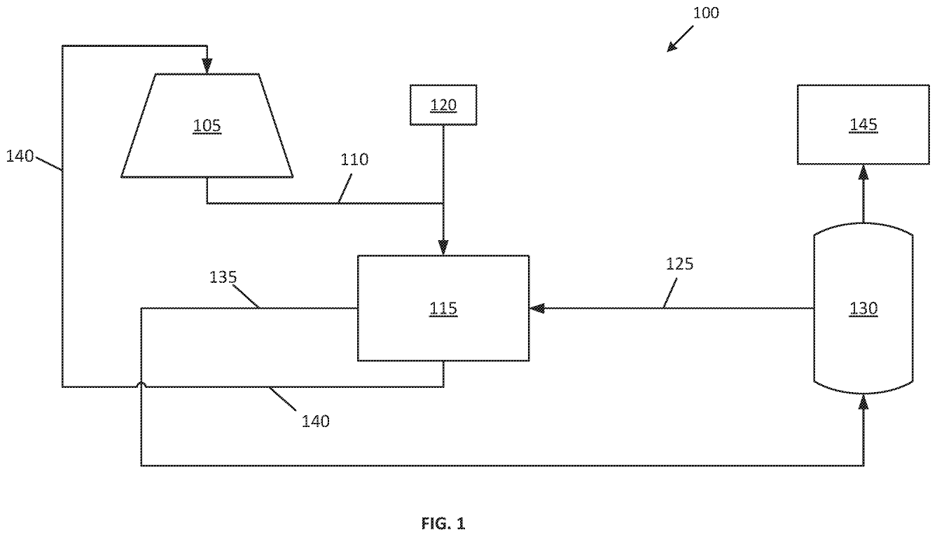

Also disclosed herein are methods for the recovery of one or more metals from a metal-containing aqueous solution (e.g., a leaching solution). A typical solvent extraction process 100 is depicted in FIG. 1. According to embodiments, a metal ore (e.g., containing copper and iron metal values) is subjected to a leaching process (e.g., a heap leaching process) where an aqueous lixiviant (e.g., sulfuric acid) is sprayed onto a heap of the ore. The lixiviant absorbs metals as it percolates down through the ore and a pregnant leaching solution (PLS) 110 exits the leaching process. The PLS 110 enters a solvent extraction system 115. The reagent compositions 120 as described herein are added to the PLS 110 using either a batch or a continuous method. In the solvent extraction system 115, the PLS 110 comes into contact with a metal-free organic solution 125 that is typically supplied by a stripping process 130. In the solvent extraction system 115, the organic solution 125 absorbs dissolved metals from the PLS 100. The reagent compositions 120 enhance this process by forming complexes with target metals in the PLS 110 where such complexes will be preferentially absorbed by the organic phase 125 in the solvent extraction system 115. A metal-loaded organic solution 135 exits the solvent extraction process 115 and is directed to the stripping process 130. An aqueous raffinate 140, which is the metal-depleted leaching solution, is recycled to the leaching process 105. During the stripping process 130, the metal is absorbed from the metal-loaded organic solution 135 and sent to an electrowinning process 145 where the metal is collected.

The one or more metals recovered by the methods described herein can include, but are not limited to, copper, molybdenum, uranium, rare earth metals (scandium, yttrium, lanthanum, cerium, praseodymium, neodymium, promethium, samarium, europium, gadolinium, terbium, dysprosium, holmium, erbium, thulium, ytterbium, and lutetium) and combinations thereof. In one embodiment, the metal is copper.

In one or more embodiments, the method includes contacting the metal-containing aqueous solution with an organic phase comprising a water immiscible solvent and a reagent composition comprising a mixture of oximes comprising at least one standard oxime and at least one 3-methyl oxime. The method can include separating the resultant metal-pregnant organic phase from the resultant metal-barren aqueous phase and recovering metal values from the metal-pregnant organic phase. In one embodiment, copper from a copper-containing aqueous solution is recovered, which includes contacting the copper-containing aqueous solution with an organic phase comprising a water immiscible solvent and a reagent composition of the type described herein. Another embodiment is where the recovered metal is selected from uranium, molybdenum, cobalt, copper, nickel and combinations thereof.

In further embodiments, also described herein are methods of separating copper from iron in an aqueous solution containing dissolved copper and iron values. In one embodiment, the method includes contacting the aqueous solution with a water-immiscible organic solution comprising a mixture of oximes. In certain embodiments, the water-immiscible organic solution comprises a hydrocarbon solvent and a mixture of oximes.

The feedstock solution containing dissolved metal values is contacted with the water-immiscible organic solution comprised of a hydrocarbon solvent as described herein and reagent composition comprising the mixture of oximes for a period of time sufficient to allow the oximes to form complexes with the iron and copper ions. The feedstock can be contacted by the organic solution in any manner that brings the two immiscible phases together for a period of time sufficient to allow the oximes in the reagent composition to form a complex with the metal ions. This includes shaking the two phases together in a separatory funnel or mixing the two phases together in a mix tank as described in U.S. Pat. No. 4,957,714, the entire contents of which are incorporated herein by reference.

In further embodiments, described herein are methods of recovering metal from an aqueous solution. The method can include contacting an aqueous solution containing at least two metals selected from molybdenum, cobalt, nickel, zinc and iron with an organic solvent and an oxime-containing reagent composition comprising a mixture of oximes as described herein.

### EXAMPLES

The following examples illustrate the effect of leaching aids according to various example aspects of the disclosure. While the examples described below used copper containing ore, it is to be understood that the examples are illustrative of any metal-containing ore body.

### Example 1

### Improved Degradation

Five 3 liter, 3-necked, jacketed, baffled round bottom flasks were set up to run at 55° C. in order to determine the difference in degradation rates between various oximes. Oximes were obtained by BASF®. Each reagent composition was 30 v/v % oximes in Orform® SX-12 organic solvent. The aqueous solution was a pregnant leach solution (PLS) from a Rapid Oxidative Leach process. The degradation pots were constantly mixed at 400 rpm. A volume of 1250 mL of the aqueous solution and 1250 mL of the reagent composition were mixed together in each degradation pot using a 3 inch polytetrafluoroethylene (PTFE) paddle impeller. Once per week, 25 mL of emulsion was taken from each pot and allowed to separate. The aqueous solution was set aside, while the reagent composition was max loaded using an aqueous QC feed. The maximum load was performed by contacting the reagent composition with a fresh QC feed, four times for three minutes. The maximum loaded organic was analyzed for copper by atomic absorption. The remaining organic solution was stripped with 250 g/L H₂SO₄, and the oxime ratio was determined by gas chromatography.

### Example 2

### Method of Preparing a Reagent Composition

BASF's Optiform process was used to determine a set of parameters for a reagent composition containing a mixture of oximes dissolved in an organic solvent. Reagent 1 (NSAO), Reagent 2 (HNAO) and Reagent 3 (3-MNSAO) listed in Table 1 were used in the following ratios; 0≤Reagent 1≤50%, 20%≤Reagent 2≤80%, and 20%≤Reagent 3≤80%. All isotherms were measured at 21° C. A solution containing 45 g/L Cu and 170 g/L sulfuric acid at 35° C. were used to determine the strip point. Isotherm data was then input into a Solvent Extraction (SX) modeling program BASF's Isocalc) to determine metal recoveries. The organic to aqueous (O:A) ratio used in the SX modeling program for group was determined by first running the modeling program for each combination of reagents and determining which O:A gave 92% recovery for a particular blend. That O:A ratio was then used to determine the remaining data for each group. The Optiform results for a three-component reagent composition at low pH, intermediate pH and high pH are shown in FIGS. 2A-2C, respectively.

The resulting Optiform charts in FIGS. 2A-C show the sensitivity of oxime formulations to changes in pH. By adjusting the concentration of each oxime in the mixture of oximes in the reagent composition, the recovery could be increased for a specific reagent formulation as compared to a comparative (i.e., existing) reagent or the amount of the reagent composition needed to reach a specific recovery could be reduced as compared to the amount needed of a comparative reagent. Based on the results of the Optiform process, McCabe Thiel isotherms were generated using formulations that were in “red” zones 205 of the Optiform charts signifying formulations that were expected to provide the highest recoveries.

FIG. 2D is a graph of the reagent concentration (v/v %) as a function of metal recovery (% by weight of total dissolved metal in the PLS) for a comparative reagent 215 and reagent compositions 210, 216 according to embodiments described herein based on the low pH Optiform data from FIG. 2A. The comparative reagent is 5-nonylsalicylaldoxime (NSAO) dissolved in trideconal. FIG. 2D illustrates the advantage of preparing a reagent composition having a particular synergistic mixture of oximes for a specific solvent extraction process (e.g., a customer wants to improve metal recovery or reduce costs of its solvent extraction process) operating at a low pH. Two advantages that can be obtained are (1) an increased recovery of metal from the PLS at a specific concentration of the mixture oximes in the reagent composition (i.e., for a target concentration, improving the recovery of metals) and/or (2) a reduced concentration of the reagent composition (i.e., reducing the amount of reagent composition consumed) needed to maintain a specific recovery.

As shown in FIG. 2D, if the aim is to maintain a 92% metal recovery for an existing low pH SX process, it could be accomplished with 5.9 v/v % of the reagent composition 210 versus 8.1 v/v % of the comparative (i.e., existing) reagent 215 having a different formulation. If the aim is to increase the metal recovery without changing reagent concentration, the recovery could be improved from 88.2% for the comparative reagent 215 to 92.5% for the reagent composition 210 at a reagent concentration of 6.2 v/v %. In either case, by selecting a synergistic mixture of oximes for the reagent composition 210, 216, there is an increased benefit in the low pH SX process.

FIG. 2E shows the reagent concentration as a function of metal recovery for a comparative reagent 215 and reagent compositions 211, 216 according to embodiments described herein based on the intermediate pH Optiform data from FIG. 2B. The comparative reagent is a mixture of 2-hydroxy-5-nonyl-benzaldehyde oxime (branched), 4-nonyl-phenol (branched), an organic ester, toluene and petroleum distillates. As shown in FIG. 2E, the metal recoveries for reagent composition 211 having the mixture of oximes as compared to the comparative (i.e., existing) reagent 215 provided modest improvements in concentration and metal recovery at an intermediate pH. In such cases, the reagent composition may be analyzed for any improvements in copper to iron (Cu:Fe) selectivity and/or degradation resistance which, if favorable, may justify switching to the new reagent composition.

### Example 3

### Degradation Resistance

The degradation, or volume percent reagent as a function of days, was determined for each of Reagent 1, Reagent 2, Reagent 3 and Reagent 4 set forth in Table 1. Each reagent was mixed with an electrolyte solution (or harsh PLS solution) at about 10 v/v % reagent. These experiments allowed for an acceleration of the degradation expected in the SX process and compared the degradation resistance of oximes, but did not provide an absolute rate of degradation for an oxime in the SX process.

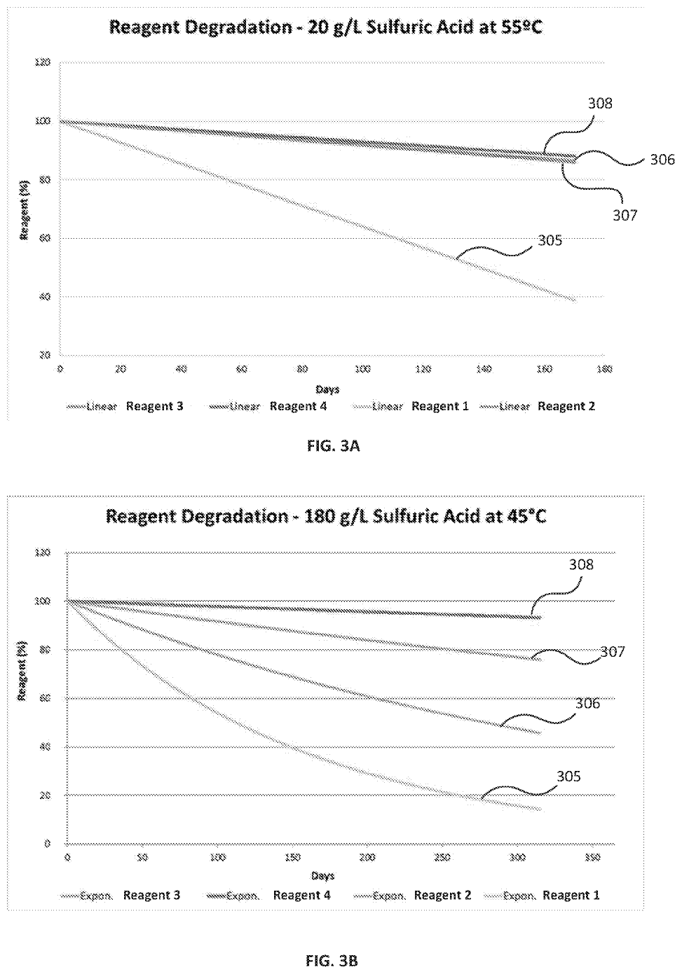

FIG. 3A shows the reagent degradation for each of the reagents when mixed with harsh PLS conditions: 20 g/L sulfuric acid and about 20 g/L copper at 55° C. stirring continuously. As shown in FIG. 3A, HNAO (Reagent 2) 306, 3-MNSAO (Reagent 3) 307 and 3-MHNAO (Reagent 4) 308 have similar degradation trends. The NSAO (Reagent 1) 305, however, degraded at a far faster rate than the other oximes. Without being bound by any particular theory, it is believed that the NSAO degraded at a faster rate because acid catalyzed hydrolysis of oximes requires a nucleophile, in this case water, to attach to the imine carbon. The NSAO has a pendant hydrogen, while the HNAO has a pendant methyl group, which causes a substantial amount of steric hindrance. The HNAO, therefore, may be more resistant to hydrolysis because the water molecule is less likely to be able to attack the imine carbon. In the case of the 3-MNSAO and the 3-MHNAO, the substitution of chemical moieties onto the phenoxy ring reduce the oxime functionality and thus, water is less likely to be able to reach the imine carbon, making these compounds more hydrolytically stable.

FIG. 3B shows the reagent degradation for each of the reagents when mixed with an electrolyte solution of 180 g/L sulfuric acid at 45° C. stirring continuously. As shown in FIG. 3B, the relative degradation resistance of each of the oxime reagents 305, 306, 307 and 308 is more distinct. The order of degradation resistance is: 3-MHNAO (Reagent 4) 308, 3-MNSAO (Reagent 3) 307, HNAO (Reagent 2) 306 and NSAO (Reagent 1) 305. Without being bound by any particular theory, it is believed that the chemical structures of the oximes discussed in the preceding paragraph have an even greater effect on the degradation resistance of Reagents 3 and 4 over Reagents 1 and 2 and of the ketoximes (Reagents 2 and 4) over the aldoximes (Reagents 1 and 3) in the electrolyte solution.

In accordance with embodiments herein, by substituting a higher percentage of a standard aldoxime with a 3-methyl aldoxime and in combination with at least one of the standard ketoxime or 3-methyl ketoxime, it is possible to formulate a mixture of oximes that increase degradation resistance. A mixture of a standard aldoxime and a 3-methyl oxime was prepared and the degradation resistance of the resulting mixture was evaluated. The standard aldoxime and the 3-methyl aldoxime have different pKa values and the strength of the 3-methyl aldoxime (i.e., the extraction versus stripping power) is far greater than for the standard aldoxime. When using high concentrations of standard aldoxime versus the standard ketoxime, the net transfer is benefitted from having some 3-methyl aldoxime. In addition to improved degradation resistance, the reagent compositions can also have improved recoveries over the comparative reagent.

### Example 4

### Copper to Iron Selectivity

As set forth in Table 1, Reagents 3 and 4 have a very high selectivity of copper over iron as compared to Reagents 1 and 2. Mixtures of Reagents 3 and 4 expectedly have a very high selectivity of copper over iron as compared to Reagents 1 and 2. Mixtures of Reagents 3 and/or 4 with at least one of Reagents 1 or 2 have a selectivity that is more moderate. However, even moderate selectivity increases can affect the efficiency of wash stages and the bleed from electrolyte systems used to control iron in the tankhouse.

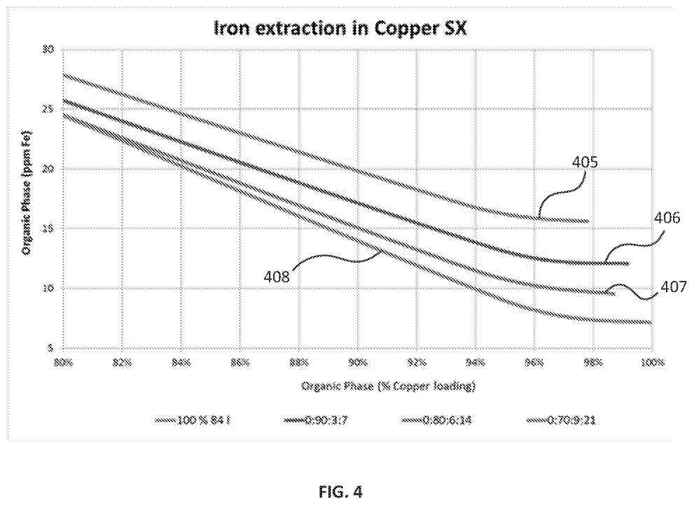

FIG. 4 shows the concentration (ppm) of iron in the organic phase as a function of mass percent of copper loading in the organic phase for a comparative reagent 405 and reagent compositions 406, 407, 408. The comparative reagent 405 contains only HNAO. The reagent compositions contain mixtures of HNAO (Reagent 2), 3-MNSAO (Reagent 3) and 3-MHNAO (Reagent 4) according to embodiments herein at the following respective weight ratios: 90:3:7 (406), 80:6:14 (407) and 70:9:21 (408). FIG. 4 shows that the Cu:Fe selectivity increased as the amount of HNAO in the reagent composition decreased and the amount of the 3-MNSAO and 3-MHNAO in the reagent composition increased. At about 94% copper loading, the iron loaded on the organic for the comparative reagent was 17 mg/L iron while the 70 wt % HNAO, 9 wt % 3-MNSAO and 21 wt % 3-MHNAO reagent composition 408 had an organic iron concentration of 9 mg/L. In this case, the synergistic mixture of the oximes in reagent composition 408 increased the copper selectivity results with approximately half the iron loading of the comparative reagent 405. In addition to improved copper selectivity, the reagent compositions can also have improved degradation resistance over the comparative reagent.

### Example 5

### Comparison of Reagent Compositions in Different Solvent Extraction Processes

Reagent compositions 506 and 507 were prepared according to embodiments herein and contained a mixture of Reagent 2, Reagent 3 and Reagent 4 from Table 1 at various weight ratios. The reagent compositions 506 and 507 were compared to a comparative reagent 505 as used in an existing customer solvent extraction process. The comparative reagent was a mixture of HNAO and NSAO at a ratio of 4.8:5.6 and a concentration of 984N. The reagent compositions contained Reagent 2, Reagent 3 and Reagent 4 at the following respective weight ratios: 0.2:0.2:0.6 (506) and 0.3:0.35:0.35 (507). The reagents were used to recover copper during a solvent extraction process.

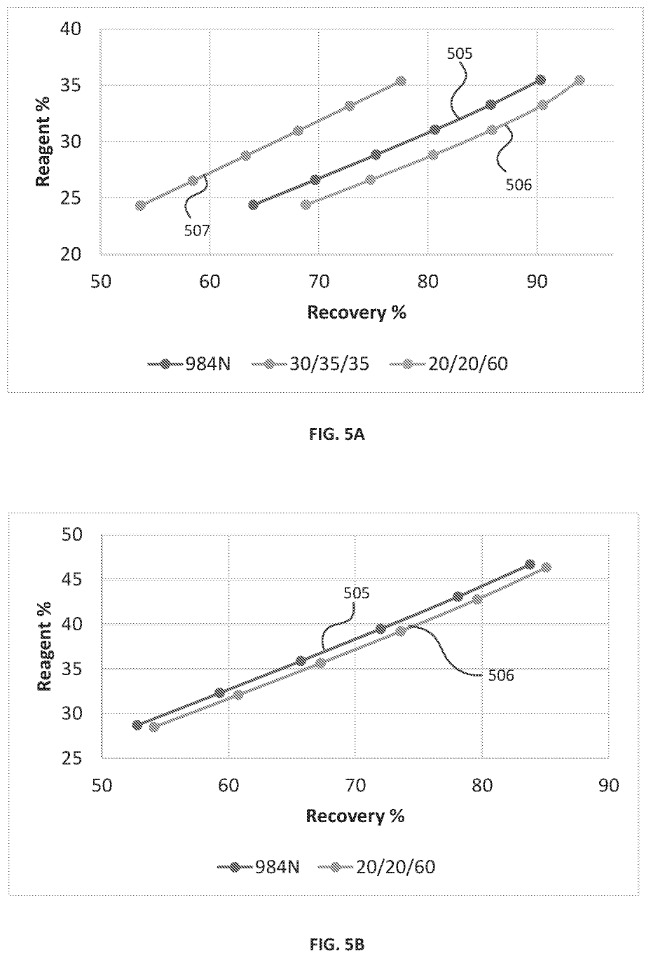

FIGS. 5A-5B show the reagent concentration (v/v %) as a function of the metal recovery (wt %) for each of the reagent compositions 506 and 507. Each of FIGS. 5A and 5B represent a different customer's solvent extraction process.

For the solvent extraction process relating to FIG. 5A, reagent composition 506 demonstrated a moderate increase in metal recovery at all reagent concentrations and a moderate decrease in reagent concentration at all metal recoveries as compared the comparative reagent 505. Reagent 507 did not show an improvement over the comparative reagent 505, but the selectivity and degradation characteristics of reagent compositions 507 should also be considered before determining whether this reagent composition provides an ultimate benefit.

Regarding the solvent extraction process relating to FIG. 5B, reagent composition 506 demonstrated a small increase in metal recovery at all reagent concentrations and a small decrease in reagent concentration at all metal recoveries as compared the comparative reagent 505. For the solvent extraction process relating to FIG. 5B, reagent composition 506 had similar performance to the comparative reagent 505.

### Example 6

### Pilot Plant Evaluation

When evaluating a dynamic extraction system, the desired target is the concentration of copper in the raffinate. This is initially determined using mathematical modeling in (small scale laboratory) static equilibrium testing (Isotherm modeling). If the data collected from a circuit or pilot plant falls within 2-3% of this calculated value, then small scale lab testing is appropriate. While circuit testing for standard reagents (e.g., NSAO) provided values within 2% of the static testing target, the 3-MNSAO and 3-MHNAO reagents fell outside of the 2-3% range. Therefore, a pilot plant was utilized to verify the isotherm results of these reagent systems.

Isotherms for various formulations were compared in the pilot plant. The following reagents were evaluated: (1) NSAO in trideconal, (2) a mixture of 20 mol % NSAO, 60 mol % HNAO, 20 mol % 3-MNSAO and 0 mol % 3-MHNAO and (3) a mixture of 60 mol % NSAO, 30 mol % HNAO, 10 mol % 3-MNSAO and 0 mol % 3-MHNAO. The comparative results for Reagents (1) and (2) are shown in Table 2.

TABLE 2
Pilot Plant Results

| Average | Average | Average | Average | Average | Average |
|---|---|---|---|---|---|
| of 6 | of 1 | of 7 | of 3 | of 4 | of 2 |
| samples | sample | samples | samples | samples | samples |
| Lean Electrolyte -27.5 gpL Cu (plant lean electrolyte) | | | | | |
| Reagent | | | | | |
| Reagent (2) | | | | | Reagent (1) |
| Parallel Extractor Continuity | | | | | |
| Organic | Aqueous | Organic | Aqueous | Organic | Aqueous |

Cu (gpL)

| | | | | | | |
|---|---|---|---|---|---|---|
| Amount of | 0.096 | 0.092 | 0.077 | 0.061 | 0.085 | 0.060 |
| Cu Leaving | | | | | | |
| Plant | | | | | | |
| (Aqueous) | | | | | | |
| Amount of | 0.054 | 0.050 | 0.054 | 0.056 | 0.055 | 0.036 |
| Cu Leaving | | | | | | |
| Plant2 | | | | | | |
| (Aqueous) | | | | | | |
| Cu Recovered | 91% | 92% | 92% | 93% | 92% | 94% |
| (wt %) | | | | | | |
| Net Transfer | 1.279 | 1.299 | 1.323 | 1.353 | 1.317 | 1.315 |
| from Organic | | | | | | |
| to Aqueous | | | | | | |

The comparative results between Reagent (1) and Reagent (3) are shown in Table 3.

TABLE 3
Pilot Plant Results

| Average of | Average of | Average of | Average of |
|---|---|---|---|
| 6 profiles | 1 profile | 7 profiles | 3 profiles |
| LE ~34.5 gpL Cu (Adjusted plant LE) | | | |
| Reagent | | | |
| Reagent (1) | | Reagent (3) | |
| Parallel Extractor Continuity | | | |
| Organic | Aqueous | Organic | Aqueous |

Cu (gpL)

| | | | | |
|---|---|---|---|---|
| Amount of | 0.079 | 0.060 | 0.080 | 0.057 |
| Cu Leaving | | | | |
| Plant | | | | |
| (Aqueous) | | | | |
| Amount of | 0.052 | 0.055 | 0.062 | 0.058 |
| Cu Leaving | | | | |
| Plant2 | | | | |
| (Aqueous) | | | | |
| Cu | 92% | 93% | 92% | 93% |
| Recovered | | | | |
| (wt %) | | | | |
| Net | 1.352 | 1.346 | 1.384 | 1.365 |
| Transfer | | | | |
| from | | | | |
| Organic to | | | | |
| Aqueous | | | | |

As shown in Table 3, Reagent (3) achieved the same recovery as Reagent (1). Based on the stage efficiency, which is the difference between the theoretical maximum recovery and the actual recovery, Reagent (1) had a slightly higher efficiency than Reagent (3). Nonetheless, the stage efficiencies of Reagent (3) are in an acceptable range.

The data as shown in Tables 2 and 3 further demonstrate that Reagent (1) would be the best modified aldoxime formulation for the plant and Reagent (3) would be the best standard blend for the plant. The overall best reagent would be the Reagent (3), which reduced iron transfer and entrainment. The testing shows that a reduction in the amount of modifier (i.e., trideconal) would help provide a more suitable formulation.

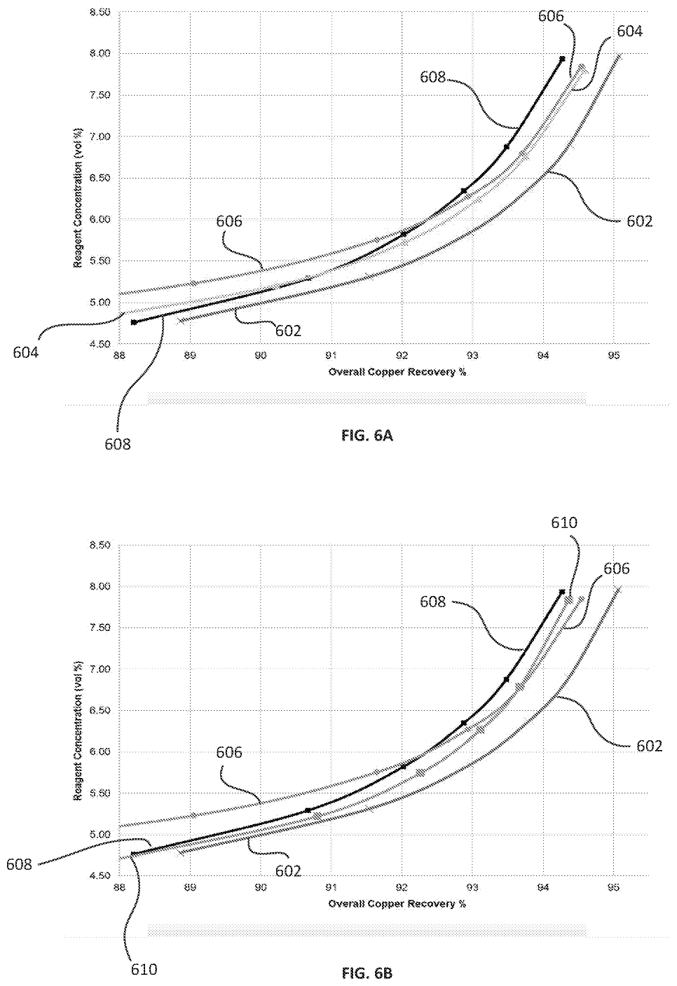

FIG. 6A plots reagent concentration (vol %) as a function of overall copper recovery (wt %) for a plant organic with its current reagent 608, with Reagent (1) 606, with a mixture of 70 mol % NSAO and 30 mol % HNAO (Reagent (4)) 604 and with Reagent (3) 602. Reagent (3) 602, consistent with embodiments described herein, outperformed 604, 606 and 608 over the entire concentration range.

FIG. 6B plots reagent concentration (vol %) as a function of overall copper recovery (wt %) for a plant organic with its current reagent 608, with Reagent (1) 606, with Reagent (3) 602 and with a mixture of NSAO and TXIB (Reagent (5)) 610. Reagent (3) 602, consistent with embodiments described herein, outperformed 606, 608 and 610 over the entire concentration range.

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Thus, the specific details set forth are merely exemplary. Particular implementations may vary from these exemplary details and still be contemplated to be within the scope of the present invention.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the invention should, therefore, be determined with reference to the appended claims, along with the full scope of equivalents to which such claims are entitled.

1. A reagent composition comprising:
a mixture of at least two oximes, at least one first oxime having a structure represented by: 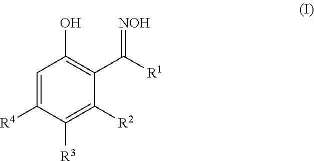
wherein R¹ is hydrogen or a C₁₋₂₂ linear or branched alkyl or alkenyl group,
R², R³ and R⁴ are each independently hydrogen, a halogen, a linear or branched C₁₋₂₂ alkyl group, OR⁶ wherein R⁶ is a C₁₋₂₂ linear or branched alkyl group, a C₂₋₂₂ linear or branched alkenyl group, a C₆ aryl group, or a C₇₋₂₂ aralkyl group, wherein at least one of R², R³ or R⁴ is not hydrogen; and
at least one second oxime having a structure represented by: 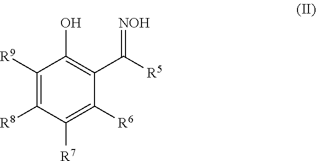
wherein R⁵ is hydrogen or a C₁₋₂₂ linear or branched alkyl or alkenyl group,
R⁶, R⁷ and R⁸ are each independently hydrogen, a halogen, a linear or branched C₁₋₂₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group, wherein at least one of R⁶, R⁷ or R⁸ is not hydrogen, and
R⁹ is a C₁₋₂₂ linear or branched alkyl or alkenyl group, and
wherein when R¹ is hydrogen, the reagent composition comprises less than about 70% by volume of the at least one first oxime,
wherein when R¹ is a C₁₋₂₂ linear or branched alkyl or alkenyl group, the reagent composition comprises less than about 85% by volume of the at least one first oxime,
wherein when R⁵ is hydrogen, the reagent composition comprises at least about 15% by volume to less than about 70% by volume of the at least one second oxime, and
wherein when R⁵ is a C₁₋₂₂ linear or branched alkyl or alkenyl group, the reagent composition comprises at least about 15% by volume of the at least one second oxime.

2. The reagent composition of claim 1, wherein the at least one first oxime has a structure selected from a group consisting of:

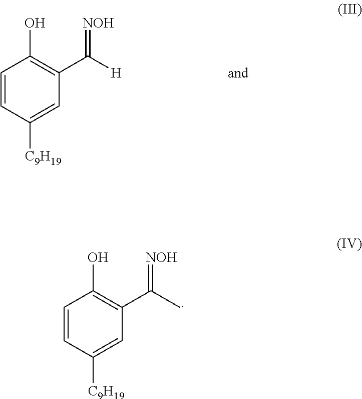

3. The reagent composition of claim 1, wherein the at least one second oxime has a structure selected from a group consisting of:

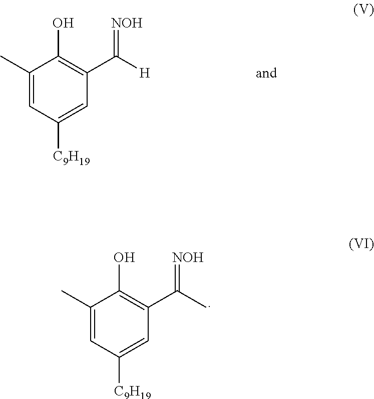

4. The reagent composition of claim 1, wherein the mixture comprises:

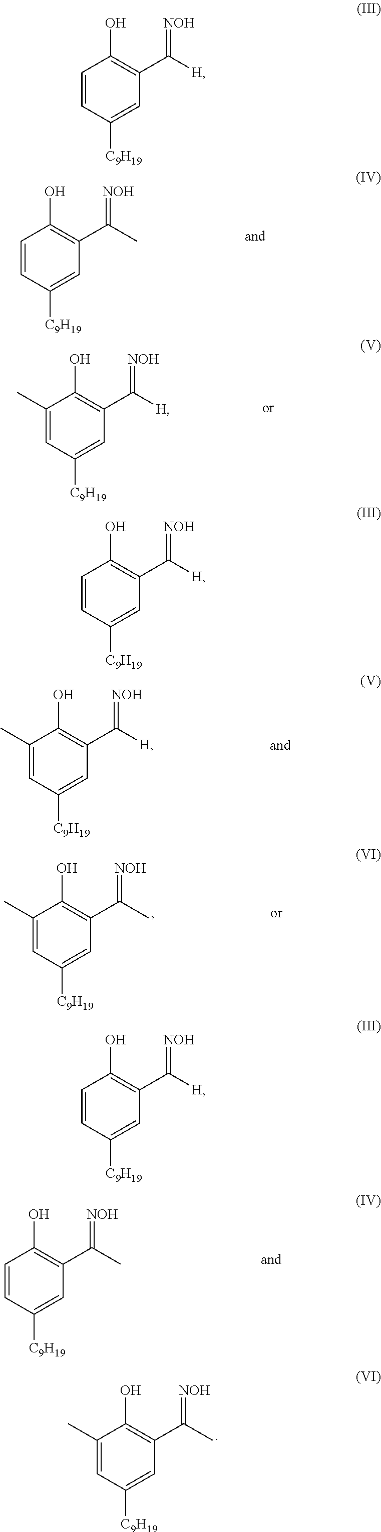

5. The reagent composition of claim 4, wherein (III) is at a concentration of about 0 wt % to about 50 wt %, (IV) is at a concentration of about 20 wt % to about 80 wt %, (V) is at a concentration of about 20 wt % to about 80 wt %, and (VI) is at a concentration of at least about 15 wt %.

6. The reagent composition of claims 1, wherein an amount of the reagent composition to provide a target recovery of metal from a process stream is less than an amount of a comparative reagent composition that does not comprise the mixture.

7. The reagent composition of claim 6, wherein the comparative reagent composition comprises a predetermined concentration of HNAO or NSAO or 3-MNSAO or 3-MHNAO, or a mixture thereof.

8. The reagent composition of claim 6, wherein the amount of the reagent composition to provide the target recovery of metal is at least about 2% by weight less than the amount of the comparative reagent composition to provide the target recovery, wherein the at least about 2% by weight is of a formulated volume of reagent (5.6 max load).

9. The reagent composition of claim 1, wherein at a target concentration, the reagent composition has a higher recovery of metal from a process stream than a comparative reagent composition that does not comprise the mixture.

10. The reagent composition of claim 9, wherein the reagent composition provides a recovery of at least about 4% by weight higher than the recovery of the comparative reagent composition.

11. The reagent composition of claim 1 comprising:

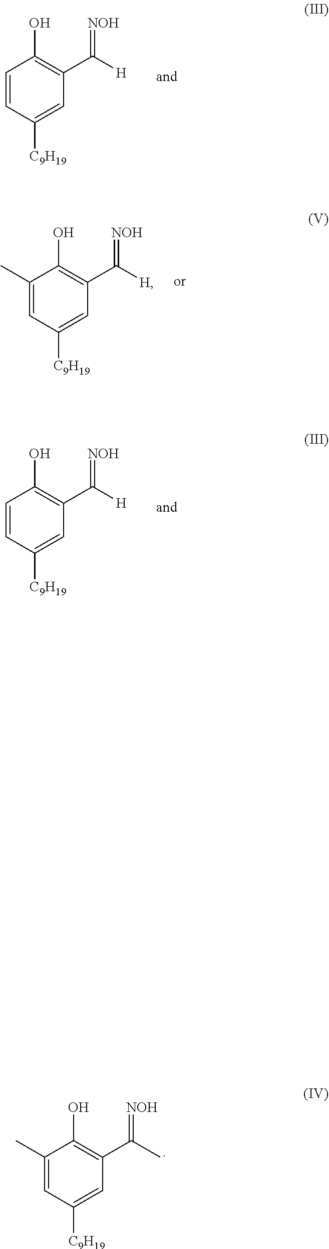

12. The reagent composition of claim 1, wherein the reagent composition has less degradation than a comparative reagent composition that does not comprise the mixture.

13. The reagent composition of claim 1 comprising:

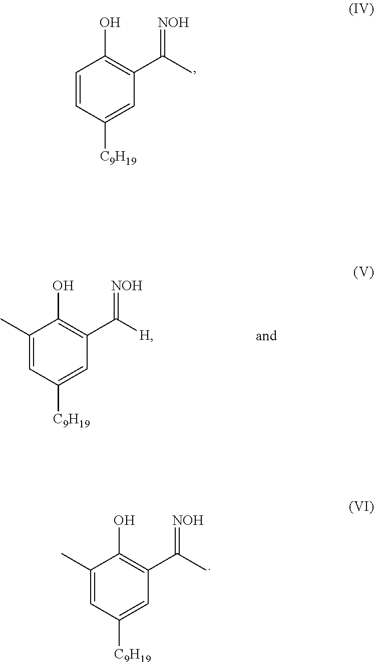

14. The reagent composition of claim 13 comprising about 10 wt % to about 70 wt % (IV), about 3 wt % to about 85 wt % (V) and about 7 wt % to about 70 wt % (VI).

15. The reagent composition of claim 1, wherein the reagent composition has a higher selectivity for copper over iron than a comparative reagent composition that does not comprise the mixture.

16. The reagent composition of claims 6, wherein the comparative reagent composition is selected from a group consisting of a standard ketoxime, a standard aldoxime, a mixture of two comparative substitutions of the first oxime and a mixture of two comparative substitutions of the second oxime.

17. A method comprising:
preparing a reagent composition comprising a mixture of at least two oximes, the mixture comprising:
at least one first oxime having a structure represented by: 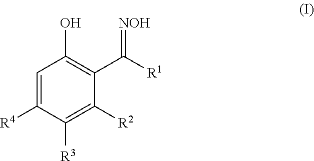
wherein le is hydrogen or a C₁₋₂₂ linear or branched alkyl or alkenyl group,
R², R³ and R⁴ are each independently hydrogen, a halogen, a linear or branched C₁₋₂₂ alkyl group, OR⁶ wherein R⁶ is a C₁₋₂₂ linear or branched alkyl group, a C₂₋₂₂ linear or branched alkenyl group, a C₆ aryl group, or a C₇₋₂₂ aralkyl group, wherein at least one of R², R³ or R⁴ is not hydrogen; and
at least one second oxime having a structure represented by: 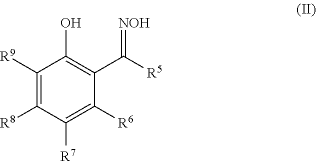
wherein R⁵ is hydrogen or a C₁₋₂₂ linear or branched alkyl or alkenyl group,
R⁶, R⁷ and R⁸ are each independently hydrogen, a halogen, a linear or branched C₁₋₂₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group, wherein at least one of R⁶, R⁷ or R⁸ is not hydrogen, and
R⁹ is a C₁₋₂₂ linear or branched alkyl or alkenyl group.

18-31. (canceled)

32. A method of recovering a metal from an aqueous solution comprising metal values, the method comprising:
contacting the aqueous solution with an organic solution comprising an organic solvent and a dissolved reagent composition, wherein the reagent composition comprises a mixture of:
at least one first oxime having a structure represented by: 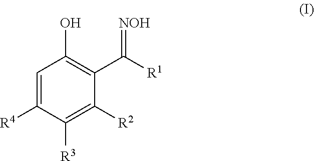
wherein R¹ is hydrogen or a C₁₋₂₂ linear or branched alkyl or alkenyl group,
R², R³ and R⁴ are each independently hydrogen, a halogen, a linear or branched C₁₋₂₂ alkyl group, OR⁶ wherein R⁶ is a C₁₋₂₂ linear or branched alkyl group, a C₂₋₂₂ linear or branched alkenyl group, a C₆ aryl group, or a C₇₋₂₂ aralkyl group, wherein at least one of R², R³ or R⁴ is not hydrogen; and
at least one second oxime having a structure represented by: 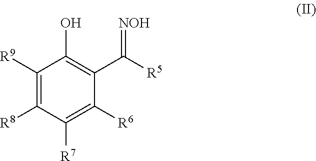
wherein R⁵ is hydrogen or a C₁₋₂₂ linear or branched alkyl or alkenyl group,
R⁶, R⁷ and R⁸ are each independently hydrogen, a halogen, a linear or branched C₁₋₂₂ alkyl group, OR¹⁰ wherein R¹⁰ is a C₁₋₂₂ linear or branched alkyl group, a C₂₋₂₂ linear or branched alkenyl group, a C₆ aryl group, or a C₇₋₂₂ aralkyl group, wherein at least one of R⁶, R⁷ or R⁸ is not hydrogen, and
R⁹ is a C₁₋₂₂ linear or branched alkyl or alkenyl group.

33-35. (canceled)

36. The method of claim 32, wherein the at least one first oxime has a structure selected from a group consisting of:

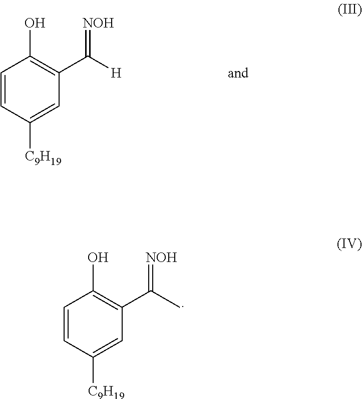

37. The method of claim 32, wherein the at least one second oxime has a structure selected from a group consisting of:

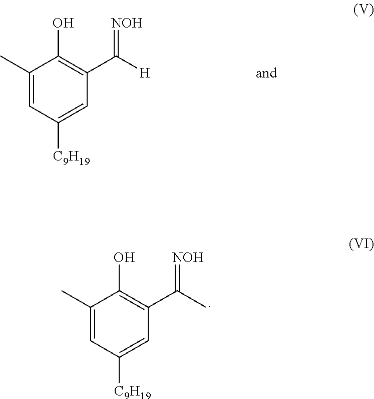

38-51. (canceled)
